(19) United States
(12) Patent Application Publication
(10) Pub. No.: US 2005/0123584 A1
(43) Pub. Date: Jun. 9, 2005

(54) Method for the treatment of food feed or agricultural products with a polyene antifungal compound

(76) Inventors: Martinus Johannes Maria Warmerdam, Rijswijk (NL); Ferdinand Theodorus Josef Rijn Van, Delft (NL); Jacobus Stark, Rotterdam (NL)

Correspondence Address: MORRISON & FOERSTER LLP, 3811 VALLEY CENTRE DRIVE, SUITE 500, SAN DIEGO, CA, 92130-2332, US

(21) Appl. No.: 10/499,627

(22) PCT Filed: Dec. 18, 2002
(86) PCT No.: PCT/EP02/14815

(30) Foreign Application Priority Data
Dec. 21, 2001 (EP) 01205118.1

(51) Int. Cl. A61K 31/7048

(52) U.S. Cl. 424/442; 514/28

(57) ABSTRACT

The present invention discloses a method for the treatment of a food, feed or agricultural product with a dry powder composition comprising a polyene antifungal compound, the method comprising:—adding the dry powder composition to the food, feed or agricultural product;and—adding an aqueous composition to the food, feed or agricultural product.

### FIELD OF THE INVENTION

The present invention relates to a method for the treatment of food, feed or agricultural products with a dry powder composition comprising a polyene antifungal compound.

The prevention of mould growth is an important topic to the food, feed and agricultural industry, especially in the food industry. Fungal spoilage can lead to serious economic losses. Some foods can be considered a good substrate for fungal growth. Cheese is an example of such a product. Apart from the negative appearance of fungal growth on cheese, fungal spoilage is also a health risk. Some mould species produce mycotoxins, which may penetrate into the product (J. C. Frisvad & U. Thane; “Mycotoxin production by food-borne fungi” in Introduction to food-borne fungi, 4^{th} edn. (ed. R. A. Samson et al.), 1995, 251-260). Therefore, superficial removal of moulds gives no guarantee of safety to the consumer.

For more than 30 years, natamycin has been used to prevent growth of moulds and yeasts on cheeses and sausages. Natamycin is on the market under the brand name of Delvocid®, a powder composition containing 50% (w/w) of natamycin.

Food products can be treated with natamycin in different ways. Natamycin can for example be added to the polymer dispersion that is applied to the cheese rind as a coating (C. B. G. Daamen & G. van den Berg “Prevention of mould growth on cheese by means of natamycin” Voedingsmiddelentechnologie, 1985, 18 (2), 26-29)

Alternatively, food products such as cheeses and sausages can be treated with a suspension of natamycin in water by dipping or spraying (C. B. G. Daamen & G. van den Berg “Prevention of mould growth on cheese by means of natamycin” Voedingsmiddelentechnologie, 1985, 18 (2), 26-29; H. A. Morris & H. B. Castberg “Control of surface growth on blue cheese using pimaricin” Cultured Dairy Products Journal,1980, 15 (2), 21-23; P. Baldini, F. Palmia, R. G. Raczynski, M. Campanini, “Use of pimaricin for preventing mould growth on Italian cured meat products”, Industria Conserve, 1979, 54 (4), 305-307; R. A. Holley, “Prevention of surface mould growth on Italian dry sausage by natamycin and potassium sorbate”, Appl. Environ. Microbiol., 1981, 41 (2).

Spraying of a suspension of natamycin can be applied for the treatment of shredded cheese, a cheese product which is very sensitive to fungal spoilage. The production of shredded cheese is as follows: cheese, e.g. Mozarella, pizza cheese, or Cheddar is shredded and conveyed to a revolving tumbler. An anti-caking agent, for example cellulose (microcrystalline or powdered), starch or modified starch is metered onto the cheese in the first part of the tumbler. Cellulose forms a film around the cheese particles which prevents caking of the shredded cheese. At the end of the tumbler a suspension of natamycin (e.g. Delvocid®) is sprayed onto the shredded cheese. The spray nozzles deliver a fine spray or mist of the fungicide onto the cheese. Finally, the cheese empties onto a conveyor to be transported to the filling equipment.

The natamycin suspension is prepared by mixing Delvocid® and water, mostly at the ratio from 2 to 10 grams of Delvocid® to 1 litre of water. The suspension is stored in a liquid container. To prevent sedimentation of the suspension a small amount of the suspension, which is pumped under pressure to spray nozzles, is recirculated to the liquid container via a jet agitator. Alternatively, a stirring device can be used to prevent sedimentation. Mostly approximately 6 to 8 litres of Delvocid® suspension is sprayed onto 1000 kg of cheese. Mostly this treatment is effective to prevent fungal spoilage.

Another way to apply natamycin is in the form of a solid dry powder composition containing natamycin and an anti-caking agent, in particular cellulose. Said solid composition may be added to prevent fungal growth on shredded cheese as disclosed in AU 734 084. The solid dry powder composition replaces both the cellulose powder composition and the aqueous natamycin suspension. The amount of natamycin in the solid powder composition may be up to 2% (w/w). Preferably, the amount of natamycin is between 0.02 and 0.5% (w/w). The solid powder composition can be prepared by mixing natamycin with the anti-caking agent, e.g. cellulose, by using for example a tumbler or a convective mixer. The solid powder composition can also be prepared in such a way that a complex between the anti-caking agent and natamycin is formed, e.g. by linking the compounds together. This can be achieved by dissolving both natamycin and cellulose in an aqueous solution by increasing, and subsequent neutralising the pH, which is followed by drying. All these steps are performed using methods known in the art. The natamycin can also be added at any suitable step in the production process of the anticaking agent.

The solid dry powder composition comprising the natamycin can be handled and applied in the same way as the anti-caking, agent is applied by methods known in the art. The resulting concentration of natamycin added to shredded cheese as a solid powder. composition, lies within the same range as when natamycin has been added as a suspension, which may vary between 2 and 40 ppm of natamycin. The optimal final concentration varies with the type of cheese, the humidity and the production method of the shredded cheese. To prevent fungal growth on other food, feed or agricultural products, the final concentration may be up to 1000 ppm natamycin.

Despite the fact that natamycin may effectively protect food, feed or agricultural products against fungal growth at given concentrations, there is always a need for improvement of the treatment method leading to a more effective protection of said products, which may result in the use of lower concentrations of natamycin.

### SUMMARY OF THE INVENTION

The present invention can provide an improved protection against the development of moulds and yeasts on food, feed or agricultural products.

The present invention provides:
(1) A method for the treatment of food, feed or agricultural products with a dry powder composition comprising a polyene antifungal compound, the method comprising:
   adding the dry powder composition to a food, feed or agricultural product; and
   adding an aqueous composition to the food, feed or agricultural product.
(2) A method for the treatment of a food, feed or agricultural product, the method comprising adding an aqueous composition to the food, feed or agricultural product which is previously treated with a dry powder composition comprising a polyene antifungal compound.
(3)Use of water to improve the protection against the development of fungal growth on a food, feed, or agricultural product which has been treated by a polyene antifungal compound.

### DETAILED DESCRIPTION OF THE INVENTION

The steps of adding the dry powder composition and the aqueous composition may be performed in any order. Preferably, the dry powder composition is added before the aqueous composition is added. Both compositions may be added by methods known in the art.

The amount of the aqueous composition added to the food, feed or agricultural products may be between 0.01% and 5% (v/w), preferably said amount is between 0.5% and 2% (v/w).

Any food, feed or agricultural product may be treated with an antifungal compound according to the method of the present invention. Examples of such products are cheese, shredded cheese, grain, animal feed (such as feed for dogs, cats, horses, cows, pigs, poultry and birds), seeds, peanuts and flower bulbs.

In a preferred embodiment, the aqueous composition as used in the method according to the present invention is water or is an aqueous solution, an aqueous emulsion of an aqueous dispersion. Preferably, said aqueous composition comprises an antimicrobial compound, for example an antifungal or antibacterial compound.

In another embodiment of the present invention, the antifungal compound in the aqueous composition and/or the dry powder composition is a polyene antifungal compound, preferably said antifungal compound is natamycin.

The optimal final concentration of natamycin added according to the method of the present invention, may vary with the type of food, feed or agricultural product. Said concentration may be between 2 and 1000 ppm natamycin, preferably said concentration is between 2 and 500 ppm natamycin, more preferably said concentration is between 2 and 100 ppm natamycin. To prevent fungal growth on shredded cheese, said optimal final concentration may be between 2 and 40 ppm natamycin, preferably said concentration is between 3 and 20 ppm natamycin, more preferably said concentration is between 4 and 10 ppm natamycin.

Any ratio wherein natamycin is added, i.e. in a dry powder composition and in an aqueous composition, may be applied. Suitable ratios of natamycin added in a dry powder composition: aqueous composition are in the range from 10%: 90% to 100% 0%.

The dry powder composition comprising a polyene antifungal compound may be any known dry powder composition, for example the commercial product Delvocid®, in case the antifungal compound is natamycin.

The dry powder composition comprising a polyene antifungal compound may also comprise an anti-caking agent. Any anti-caking agent may be used. Examples of suitable anti-caking agents are microcrystalline or powdered cellulose, starch, modified starch, sodium-, magnesium-, potassium-, and zinc silicate, silicium-dioxide, kaolin, talc, potassium and magnesium carbonate, phosphates (di, tri and polyphosphates including sodium, potassium and calcium salts). Also commercial products containing cellulose, such as Keycel or Floam® may be used as anti-caking agent. Further, any combination of anti-caking agents may be used.

The aqueous composition comprising an antifungal compound, e.g. natamycin, may be prepared by mixing a dry formulation, e.g. Delvocid®, in water using methods known in the art. Preferably a natamycin suspension for spraying is prepared by using a stock suspension of natamycin as described in EP 0 678 241. This stock suspension is a chemically, physically and microbially stable concentrated suspension of natamycin, which provides a convenient stock for the easy and reproducible dosage of a fungicide to food, feed and agricultural products. Apart from natamycin, the suspension comprises a thickening agent, for example xanthan. The concentration of natamycin in the stock suspension may be as high as 40% (w/w), however also stock suspensions with lower concentrations of natamycin can be used. An example of such a stock suspension is the commercial product Delvocid-Sol(D. This product comprises 50 to 54% (w/w) of natamycin, 2% (wiw) of xanthan and 44 to 48% (w/w) of lactose. In the case of shredded cheese the stock suspension is diluted with water to the final concentration required for the treatment. This dilution step can be best executed just before adding the natamycin suspension to the shredded cheese.

In case the polyene antifungal compound is natamycin, also complexes of natamycin (e.g. with proteins) and salts of natamycin (e.g. the calcium salt) may be used in the method according to the present invention. Further, the dry and aqueous compositions may also contain components, which are already present in a natamycin preparation. For example, when Delvocid-Instant® or Natamax® is used, lactose will also be present in the final composition.

The following Examples are for illustrative purposes only, and are not to be construed as being limitative to the invention.

### EXAMPLES

### Example 1

Cheddar cheese was shredded using methods known in the art and one of the following formulations of natamycin and cellulose was added to the shredded cheese:
A) Control, no addition of natamycin;
B) 2% (w/w) cellulose, followed by spraying with 1% (v/w) of an aqueous natamycin suspension containing 1000 μg/ml (final concentration of natamycin is 10 μg/g of cheese);
C) 2% (w/w) of cellulose, blended with 500 μg natamycin/g (final concentration of natamycin is 10 μg/g of cheese);
D) 2% (w/w) of cellulose, blended with 500 μg natamycin/gram, (final concentration of natamycin is 10 μg/g of cheese), followed by spraying with 1% (v/w) of water;
E) 2% (w/w) of cellulose, blended with 250 μg natamycin/g, followed by 1% (v/w) of an aqueous natamycin suspension containing 500 μg/g (final concentration of natamycin is 10 μg/g of cheese).

After treatment the cheese was stored in plastic bags for 21 days at 4° C. Table 1 shows the presence of molds after 21 days, expressed as log colony forming units per gram of cheese (log cfu/g). The results clearly demonstrate the superiority of a combination of dry and aqueous formulated natamycin over dry or aqueous alone.

TABLE 1

| Formulation | Log cfu/g |
|---|---|
| A: Control | 3.4 |
| B: 10 ppm of natamycin added by spraying | 2.8 |
| C: 10 ppm natamycin added with cellulose | 2.0 |
| D: 10 ppm of natamycin added with cellulose, followed by | 1.7 |
| spraying water 1% (v/w) | |
| E: 5 ppm of natamycin added with cellulose, followed by | 0.6 |
| 5 ppm of natamycin added by spraying | |

### Example 2

Cheddar cheese was shredded using methods known in the art and mixed with one of the following formulations of cellulose and natamycin:
A) Control, no addition of natamycin;
B) 2% (w/w) cellulose, followed by spraying with 1% (v/w) of an aqueous natamycin suspension containing 500 μg/ml (final concentration of natamycin is 5 μg/g of cheese);
C) 2% (w/w) of cellulose, blended with 250 μg natamycin/g (final concentration of natamycin is 5 μg/g of cheese);
D) 2% (w/w) of cellulose, blended with 250 μg natamycin/g (final concentration of natamycin is 5 μg/g of cheese), followed by spraying with 1% (v/w) of water.

After mixing with one of the above-mentioned formulations, the shredded cheese was stored in plastic bags for 21 days at 4° C. Table 2 shows the presence of molds after 21 days, expressed as log colony forming units per gram of cheese (log cfu/g).

The results clearly demonstrate the positive effect of water added after treatment with cellulose blended with natamycin.

TABLE 2

| Formulation | Log cfu/g |
|---|---|
| A: Control | 3.4 |
| B: 5 ppm natamycin added by spraying | 3.2 |
| C: 5 ppm natamycin added with cellulose | 2.4 |
| D: 5 ppm natamycin added with cellulose, followed | 2.1 |
| by spraying water 1% (v/w) | |

1. A method to protect food, feed or agricultural product, from fungal growth the method comprising:
adding the dry powder composition comprising a polyene antifungal compound to the food, feed or agricultural product; and
adding an aqueous composition to the food, feed or agricultural product.

2. The method claim 1, wherein the dry powder composition is added before the aqueous composition is added.

3. A method to protect a food, feed or agricultural product, from fungal growth wherein said method comprises
adding an aqueous composition to the food, feed or agricultural product wherein said food, feed or agricultural product has been treated with a dry powder composition comprising a polyene antifungal compound.

4. The method of claim 1, wherein the aqueous composition comprises an antimicrobial compound.

5. The method of claim 4, wherein the antimicrobial compound is an antifungal or antibacterial compound.

6. The method of claim 5, wherein the antifungal compound is natamycin.

7. The method of claim 1, wherein the aqueous composition is water.

8. The method of claim 1, wherein the aqueous composition is added in an amount between 0.01% and 5% (v/w).

9. The method of claim 1, wherein the polyene antifungal compound in the dry powder composition is natamycin, a complex of natamycin, salt of natamycin or combinations thereof.

10. The method of claim 6, wherein the final concentration of natamycin added is between 2 and 1000 ppm.

11. The method of claim 1, wherein the dry powder composition comprises an anticaking agent.

12. The method of claim 11, wherein the anticaking agent is cellulose.

13. The method of claim 11, wherein the anticaking agent is starch or modified starch.

14. The method of claim 1, wherein the food, feed or agricultural product comprises cheese.

15. A product of the method of claim 1.

16. (canceled)

17. The method of claim 3, wherein the aqueous composition comprises an antimicrobial compound.

18. The method of claim 17, wherein the antimicrobial compound is an antifungal or antibacterial compound.

19. The method of claim 18, wherein the antifungal compound is natamycin.

20. The method of claim 3, wherein the aqueous composition is water.

21. The method of claim 3, wherein the aqueous composition is added in an amount between 0.01% and 5% (v/w).

22. The method of claim 3, wherein the polyene antifungal compound in the dry powder composition is natamycin, a complex of natamycin, a salt of natamycin or combinations thereof.

23. The method of claim 19, wherein the final concentration of natamycin added is between 2 and 1000 ppm.

24. The method of claim 3, wherein the dry powder composition comprises an anticaking agent.

25. The method of claim 24, wherein the anticaking agent is cellulose.

26. The method of claim 24, wherein the anticaking agent is starch or modified starch.

27. The method of claim 3, wherein the food, feed or agricultural product comprises cheese.

28. A product of the method of claim 3.

29. The method of claim 9, wherein the final concentration of natamycin added is between 2 and 1000 ppm.

30. The method of claim 22, wherein the final concentration of natamycin added is between 2 and 1000 ppm.
